(19) United States
(12) Patent Application Publication
(10) Pub. No.: US 2019/0202885 A1
(43) Pub. Date: Jul. 4, 2019

(54) TauT (taurine transporter) peptide cancer vaccine

(71) Applicant: Xiaobin Han, Cordova, TN (US)

(72) Inventor: Xiaobin Han, Cordova, TN (US)

(21) Appl. No.: 15/858,880

(22) Filed: Dec. 29, 2017

(51) Int. Cl. C07K 14/705; A61P 35/00; A61K 39/00; A61P 35/02

(52) U.S. Cl. CPC C07K 14/705; A61P 35/00; A61K 39/001102; A61K 2039/6081; A61K 2039/572; A61K 2039/575; A61P 35/02

(57) ABSTRACT

This invention provides a TauT-peptide vaccine for cancer immunotherapy and, more particularly, a method of preventing tumor carcinogenesis comprising the steps of administering to an animal and/or human subject having a precancerous precursor of various cancers, including, but not limited to, lymphoma, sarcoma, carcinoma, thymoma, and leukemia. This invention also provides a pharmaceutical preparation comprising a TauT-peptide vaccine (prophylactic and therapeutic) to prevent, suppress or inhibit spontaneous tumorigenesis in vivo. The present invention provides a safe and effective method for suppressing or inhibiting spontaneous tumorigenesis and is particularly useful for preventing subjects having elevated risk of developing p53 gene mutation associated cancers.

### CROSS-REFERENCES TO RELATED PATENT APPLICATIONS

Application No.: 62/455,517

Filing date: FEB. 6, 2017

Name of applicant: Xiaobin Han

Title of invention: TauT (taurine transporter) peptide cancer vaccine

Confirmation No. 6690

### TECHNICAL FIELD

The present invention relates to use of a unique TauT-peptide in immunogenic compositions to induce cell-mediated immunity against cancer cells in vivo.

### BACKGROUND OF THE INVENTION

Cancer afflicts approximately 1.2 million people in the United States each year. About 50% of these cancers are curable with surgery, radiation therapy, and chemotherapy. Despite significant technical advances in these three types of treatments, each year more than 500,000 people will die of cancer in the United States alone reported by National Institute of Health (NIH) 2016.

The goal of cancer treatment is to develop modalities that specifically target tumor cells, thereby avoiding unnecessary side effects to normal tissue. Immunotherapy has the potential to provide an alternative systemic treatment for most types of cancer. The advantage of immunotherapy over radiation and chemotherapy is that it can act specifically against the tumor without causing normal tissue damage. One form of immunotherapy, vaccines, is particularly attractive because they can also provide for active immunization, which allows for amplification of the immune response. In addition, vaccines can generate a memory immune response.

The possibility that altered features of a tumor cell are recognized by the immune system as non-self and may induce protective immunity is the basis for attempts to develop cancer vaccines. Whether or not this is a viable strategy depends on how the features of a transformed cell are altered. Appreciation of the central role of mutation in tumor transformation gave rise to the hypothesis that tumor antigens arise as a result of random mutation in genetically unstable cells. Although random mutations might prove immunogenic, it would be predicted that these would induce specific immunity unique for each tumor.

There is a need for new, selective and universal anticancer agents that differentiate between malignant and nonmalignant cells based on specific cell surface proteins that provide essential environment to support cancer cell proliferation. The benefits of such agents would include a higher therapeutic index and lower toxicity than conventional therapies.

TauT (also known as SLC6A6) is a sodium and chloride-depended taurine transporter with 12-transmembrane polypeptides across the cell membrane. A TauT nucleotide sequence (SEQ ID NO 1) and amino acid sequence (SEQ ID NO 2) have been cloned and registered at the National Center for Biotechnology Information (NCBI) under Accession: U16120.1 GI: 559852 and Accession: AAC50443.1 GI: 799339, respectively.

TauT transport taurine from extracellular across cell membrane into the cells. Taurine plays an essential role in maintaining the cell volume for optimal cell growth and survival. Taurine deficiency causes cell cycle S/G2 phase arrest (1).

TauT is highly expressed in a variety of tumors, especially in leukemia cells. Studies have shown that leukocytes, including monocytes, lymphocytes, neutrophils, and eosinophils have high levels of taurine (20-50 mM), which is 500-fold higher than that in plasma. Immature leukocytes acquire taurine by active uptake rather than biosynthesis, and uptake is completed during differentiation in the bone marrow. Leukemia cells express a high level of TauT. Overexpression of TauT correlates with poor response to treatment, adverse disease progression, and poor survival.

TauT (also known as SLC6A6) has been disclosed as a pharmaceutical composition for treating colon cancer (Patent Document 1). As antibodies against SLC6A6 (also known as TauT), Patent Document 1 disclosed a monoclonal antibody specifically recognizing the extracellular region comprising amino acid residues 143-216 of SLC6A6 (TauT).

The present invention is based on the finding that taurine, an osmolyte that maintains optimal cell volume during cell proliferation is essential for rapid growth of cancer cells. Thus, the rational of approaches which inhibit TauT transportation of taurine to treat cancer is apparent and novel. The present invention aims to use a polypeptide of TauT as a peptide vaccine (named TauT-peptide vaccine) to develop a unique TauT antibody (TauT-Ab) that can directly block taurine uptake by cancer cells and inhibit cancer cells proliferation in vivo. Thus, TauT-peptide vaccine may represent a new avenue for cancer immunotherapy.

The present invention aims to provide an anticancer peptide vaccine using a TauT-peptide (SEQ ID NO 3) comprising amino acid residues 313-326 of TauT to treat cancers, particular for treating p53 tumor suppressor gene mutation related cancers. The antibodies induced by TauT-peptide vaccine in vivo specifically recognize the gating region comprising amino acid residues 313-326 of the TauT (SLC6A6), through which taurine is transported into the cancer cells and promote rapid cancer development.

### BRIEF SUMMARY OF INVENTION

### Technical Problem

Immunotherapy in cancer patients aims at activating cells of the immune system specifically, especially the so-called cytotoxic T-cells (CTL, also known as “killer cells”, also known as CD8-positive T-cells), against tumor cells but not against healthy tissue. However, priming of one kind of CTL is usually insufficient to eliminate all tumor cells. Tumors are very mutagenic and thus able to respond rapidly to CTL attacks by changing their protein pattern to evade recognition by CTLs. As result, conventional immunotherapy often have no effect on many cancers. Thus, there is a need for development of a universal cancer vaccine against cancers.

### Solution to Problem

Tumour-specific and tumour-associated antigens are often derived from proteins directly involved in transformation of a normal cell to a tumor cell due to mutation of up-stream tumor suppressors and/or gain-of-function of oncogenes, hick regulate cell cycle control or apoptosis. Additionally, the proteins that is the downstream target of the oncogenes directly causative for a transformation may be upregulated and thus be indirectly tumour-associated. Such indirectly tumour-associated antigens may also be targets of a vaccination approach. TauT is a downstream target gene of p53 tumor suppressor and oncogene c-Jun (1, 2). TauT is overexpressed in many types of cancer cells. Essential is in these cases the presence of epitopes in the ammo acid sequence of the antigen, since such peptide (“immunogenic peptide”) that is derived from a tumour-associated antigen should lead to an in vitro or in vivo T-cell-response. To counter-attack the tumor evasion mechanisms, this invention aims to provide a specific universal TauT-peptide vaccine for cancer immunotherapy.

### Advantageous Effects of Invention

The present invention aims to provide a TauT-peptide vaccine, which induces an immune response against tumor cells that overexpress as antigen the taurine transporter (TauT). The antibody induced by this TauT-peptide vaccine in vivo recognizes the amino acid residues 313-326 of TauT (SLC6A6) and inhibits cancer development resulting prolonged lifespan of animals who bear the homozygous deletion of p53 tumor suppressor gene. More than 50 percent of human tumors contain a mutation or deletion of the p53 gene (3). Thus, the TauT-peptide vaccine could he used to treat cancers associated with p53 mutations. The present invention further relates to the use of TauT-peptide in immunogenic compositions to induce antibody and cell-mediated immunity against target cells, such as tumor cells, that overexpress the TauT gene.

### BRIEF DESCRIPTION OF DRAWINGS

FIG. 1A and FIG. 1B. TauT-peptide vaccination prolongs p53^{-/-} mice lifespan. To evaluate the efficacy of TauT-peptide as a cancer vaccine, p53^{-/-} (C57BL/6J background) were injected with either TauT-peptide (100 μg/kg) or vehicle only at the age of 4 weeks and boosted 2 weeks later (n=12 for each group). All mice were observed daily for signs of morbidity and the development of tumors. The p53^{-/-} control mice started to develop tumor (mainly lymphoma) at the age of 75 days (FIG. 1A). The median lifespan for p53^{-/-} control mice was 138 days (FIG. 1B), which consistent with other studies. The p53^{-/-} mice vaccinated with TauT-peptide vaccine showed 100% response to the vaccine. TauT-peptide vaccination delayed tumor develops in all vaccinated p53^{-/-} mice, which started to develop tumor (mainly lymphoma) at the age of 189 days (FIG. 1A). TauT-peptide vaccination significantly increased (1.5 fold) median lifespan of the p53^{-/-} mice from 138 days (control) to 207 days (TauT-peptide vaccinated) (FIG. 1B). This finding is the first to demonstrate that a novel peptide vaccine (TauT-peptide vaccine) against a specific cell surface protein-TauT can delay, inhibit and/or treat p53 homogenous mutation related spontaneous tumorigenesis in vivo.

FIG. 2A and FIG. 2B. TauT-peptide vaccination prolongs p53^{+/-} mice lifespan. To evaluate the efficacy of TauT-peptide as a cancer vaccine, p53^{+/-} (C57BL/6J background) were injected with either TauT-peptide (100 μg/kg) or vehicle only at the age of 4 weeks and boosted 2 weeks later (n=15 for each group). All mice were observed daily for signs of morbidity and the development of tumors. The p53^{+/-} control mice started to develop tumor (mainly osteosarcoma) at the age of 186 day (FIG. 2A). The median lifespan for p53^{+/-} control mice was 315 days (FIG. 2B), which consistent with other studies. The p53^{+/-} mice vaccinated with TauT-peptide vaccine showed 100% response to the vaccine. TauT-peptide vaccination delayed tumor develops in all vaccinated p53^{+/-} mice, which started to develop tumor (mainly osteosarcoma) and died at the age of 526 days (FIG. 2A). TauT-peptide vaccination significantly increased median lifespan of the p53^{+/-} mice from 315 days (control) to 621 days (TauT-peptide vaccinated) (FIG. 2B). This finding is the first to demonstrate that a novel peptide vaccine (TauT-peptide vaccine) against a specific cell surface protein-TauT can delay, inhibit and/or treat p53 heterogeneous mutation related spontaneous tumorigenesis in vivo.

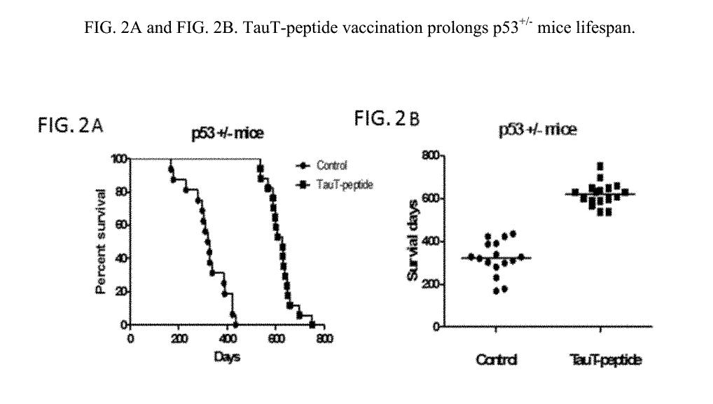

FIG. 3. Effect of the TauT-Ab on cell viability of primarily cultured thymocytes. Thymocytes were isolated from rat thymus and cultured in the RPMI-1640 medium containing 10% fetal bovine serum. Thymocytes were treated with equal amount of pre-immune IgG (control IgG) and TauT-Ab for 48 hours. Untreated thymocytes were used as normal control. Cells were counted and cell viability was calculated basis on the percentage of life cells out of totally counted cells. TauT-Ab treated thymocytes showed a ˜80% cell viability similar to that of pre-immune IgG treated thymocytes, suggesting that TauT-Ab had no effect on cell viability in normal cells (FIG. 3). The graph represents typical results of four separate experiments.

FIG. 4A and FIG. 4B. Dose- and time-dependent of TauT-Ab induced cell death of Jurkat human leukemia cells. Jurkat cells were cultured in the medium containing indicated amount of IgG and TauT-Ab for 24 hours. Dose-dependent (FIG. 4A) and time-dependent (FIG. 4B) cell viability were calculated basis on the percentage of life cells out of totally counted cells over indicated doses (0-10 μg/ml) and time points (0-72 hrs) in the presence of TauT-Ab (5 μg/ml). Untreated cells were used as control. TauT-Ab induced Jurkat cell death in dose- and time-dependent manners. The graph represents typical results of four separate experiments.

FIG. 5. Effect of the TauT-Ab on cell viability of human acute promyelocytic leukemia HL-60 cells. HL-60 cells were cultured in the RPMI-1640 medium containing 10% fetal bovine serum. HL-60 cells were treated with equal amount (10 μg/ml) of pre-immune IgG and TauT-Ab for indicated time. Untreated HL-60 cells were used as control. Cells were counted and cell viability was calculated basis on the percentage of life cells out of totally counted cells. TauT-Ab induced HL-60 cell death in time-dependent manners (FIG. 5). The graph represents typical results of four separate experiments.

FIG. 6. Effect of the TauT-Ab on cell viability of human placental carcinoma Jar cells. Jar cells were cultured in the DMEM medium containing 10% of fetal bovine serum. Jar cells were treated with equal amount (10 μg/ml) of pre-immune IgG and TauT-Ab for indicated time. Cells were counted and cell viability was calculated basis on the percentage of life cells out of totally counted cells. Untreated Jar cells were used as control. TauT-Ab induced Jar cell death in time-dependent manners (FIG. 6). The graph represents typical results of four separate experiments.

FIG. 7. Effect of the TauT-Ab on cell viability of human breast cancer MCF-7 cells. MCF-7 cells were cultured in the DMEM medium containing 10% of fetal bovine serum. MCF-7 cells were treated with equal amount (10 μg/ml) of pre-immune IgG and TauT-Ab for indicated time. Untreated MCF-7 cells were used as control. Cells were counted and cell viability was calculated basis on the percentage of life cells out of totally counted cells. TauT-Ab induced MCF-7 cell death in time-dependent manners (FIG. 7). The graph represents typical results of four separate experiments.

FIG. 8. Effect of the TauT-Ab on cell viability of normal marine fibroblast (10)1 cells. (10)1 cells were cultured in the DMEM medium containing 10% of fetal bovine serum. (10)1 cells were treated with equal amount (10 μg/ml) of pre-immune IgG and TauT-Ab for indicated time. Cells were counted and cell viability was calculated basis on the percentage of life cells out of totally counted cells. Untreated (10)1 cells were used as control. TauT-Ab had no effect on the viability of normal (10)1 cells (FIG. 8). The graph represents typical results of four separate experiments.

FIG. 9. Effect of the TauT-Ab on cell viability of human embryonic kidney 293 cells. 293 cells were cultured in the DMEM/F12 medium containing 10% of fetal bovine serum. 293 cells were treated with equal amount (10 μg/ml) of pre-immune IgG and TauT-Ab for indicated time. Untreated 293 cells were used as control. Cells were counted and cell viability was calculated basis on the percentage of life cells out of totally counted cells. TauT-Ab had no effect on the viability of normal human 293 cells (FIG. 9). The graph represents typical results of four separate experiments.

FIG. 10. Effect of the TauT-Ab on cell viability of normal renal proximal tubule LLC-PK1 cells. LLC-PK1 cells were cultured in the DMEM/F12 medium containing 10% of fetal bovine serum. LLC-PK1 cells were treated with equal amount (10 μg/ml) of pre-immune IgG and TauT-Ab for indicated time. Untreated LLC-PK1 cells were used as control. Cells were counted and cell viability was calculated basis on the percentage of life cells out of totally counted cells. TauT-Ab had no effect on the viability of normal LLC-PK1 cells (FIG. 10). The graph represents typical results of four separate experiments.

FIG. 11. Effect of the TauT-Ab on cell viability of TauT overexpression renal proximal tubule LLC-PK1 cells (pNCT-1 cells). The pNCT-1 cells were cultured in the DMEM/F12 medium containing 10% of fetal bovine serum. The pNCT-1 cells were treated with equal amount (10 μg/ml) of pre-immune IgG and TauT-Ab for indicated time. Pre-immune IgG-treated pNCT-1 cells were used as control. Cells were counted and cell viability was calculated basis on the percentage of life cells out of totally counted cells. TauT-Ab induced cell death of TauT-overexpression LLC-PK1 cells (FIG. 11). The graph represents typical results of four separate experiments.

FIG. 12A and FIG. 12B. Effect of the TauT-Ab on cell cycle of Jurkat leukemia cells. Jurkat cells were cultured in the RPMI-1640 medium containing 10% fetal bovine serum. Jurkat cells were treated with equal amount (10 μg/ml) of pre-immune IgG and TauT-Ab for 24 hours. Untreated Jurkat cells were used as control (FIG. 12A). Cell cycle was analyzed as described in the Methods. TauT-Ab induced cell cycle G2 arrest and cell death in Jurkat cells (FIG. 12B). The graph represents typical results of four separate experiments.

FIG. 13. TauT-Ab selectively kills the cells overexpressing TauT induced by Wilms' tumor gene (WT1). In this study WT1 was stably transfected into the LLC-PK1 cells and the cells were named as WT-7. Treatment of WT-7 cells with TauT-Ab inhibits cell growth and induces cells death within 24 hours. In contrast, pre-immune IgG had no effect on the cell viability of WT7 (FIG. 13), indicating that TauT-Ab selectively kills the cells which overexpress a WT1 gene. This finding is very important, since overexpression of WT1 has been found in various types of human leukemia.

### DESCRIPTION OF EMBODIMENTS

### Definitions

The following definitions are provided to facilitate understanding of certain terms used throughout this specification.

In the present invention, a “membrane” taurine transporter (TauT) protein is one expressed on the cell surface. The fourth intracellular segment (S4) of the TauT is the most highly charged intracellular segment, which is 100% identical among the taurine transporters cloned from the mammalians up to date. Studies have demonstrated that the S4 polypeptide fragment of TauT participates directly in the gating of taurine by TauT in mammalian cells, which is tightly controlled by endogenous protein kinase C (PKC) phosphorylation of serine-322 located in the S4 of TauT (4).

In the present invention, “epitopes” refer to S4 polypeptide fragment(s) having antigenic or immunogenic activity in an animal, especially in a human, or that are capable of eliciting a T lymphocyte response in an animal, preferably a human. A preferred embodiment of the present invention relates to a S4 polypeptide fragment (designated as TauT-peptide) comprising an epitope, as well as the polynucleotide encoding this fragment. A further preferred embodiment of the present invention relates to a S4 polypeptide fragment (TauT-peptide) consisting of an epitope, as well as the polynucleotide encoding this fragment. In specific preferred embodiments of the present invention, the epitope comprises a polypeptied fragment basis on the S4 polypeptide sequence. A region of a protein molecule to which an antibody can bind is defined as an “antigenic epitope.” In contrast, an “immunogenic epitope” is defined as a part of a protein that elicits an antibody response (Geysen et al., Proc. Natl. Acad. Sci. USA 81:3998-4002 (1983)). Thus, a further preferred embodiment of the present invention is an immunogenic S4 polypeptide fragment, TauT-peptide, that is capable of eliciting a T cell response when bound to the peptide binding cleft of an MHC molecule. Further embodiments of the invention are directed to pharmaceutical formulations comprising said immunogenic S4 peptide fragments (TauT-peptide) or the polynucleotides encoding them.

Fragments which function as epitopes may be produced by any conventional means (5).

The sequence of peptide epitopes known to bind to specific MHC molecules can be modified at the known peptide anchor positions in predictable ways that act to increase MHC binding affinity. Such “epitope enhancement” has been employed to improve the immunogenicity of a number of different MHC class I or MHC class II binding peptide epitopes (6). Accordingly, a further embodiment of the invention is directed to such enhanced S4 epitopes, and to the polynucleotides encoding such enhanced epitomes.

In the present invention, antigenic epitopes preferably contain a sequence of at least seven, more preferably at least nine, and most preferably between about 12 to 14 amino acids. Antigenic epitopes are useful to raise antibodies, including monoclonal antibodies that specifically bind the epitope (7).

Similarly, immunogenic epitopes can be used to induce B cells and T cells according to methods well known in the art (8). The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 9 amino acids have been shown to be sufficient to raise antibodies capable of binding to, at the very least, linear epitopes in a denatured polypeptide (e.g., in Western blotting.)

As used herein, the term “antibody” (Ab) or “monoclonal antibody” (mAb) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody (9). Thus, for some applications these fragments are preferred, as well as the products of a Fab or other immunoglobulin expression library. Moreover, antibodies of the present invention include chimeric, single chain, and humanized antibodies.

In accord with the purpose of the invention to provide improved means of cancer immunotherapy using antibodies to, the antibody specifically recognize the S4 segment of TauT was developed and evaluated using human carcinoma cells in vitro.

### EXAMPLES

Materials and Methods:

TauT-peptide (SEQ ID NO 3) was coupled to KLH with a kit (Imject SuperCarrier EDC system for Peptides; Pierce). To test TauT-peptide vaccine, p53 knockout (KO) mice were immunized with 100 μg/kg and boosted with an equal amount of antigen 2 weeks late. To develop and test TauT-Ab, wild type control mice were immunized with 100 μg/kg TauT-peptide and boosted with an equal amount of antigen 2 weeks late. Mice were bled on Days 21 and 36 to measure the antibody response. Antisera were collected on Day 49 and purified by affinity column chromatography (Pierce). Antisera were stored at −80° C. Specificity of the antibody was determined by Western blot analysis (10).

### Example 1

TauT-peptide Vaccination Prolongs p53 KO Mice Lifespan In vivo

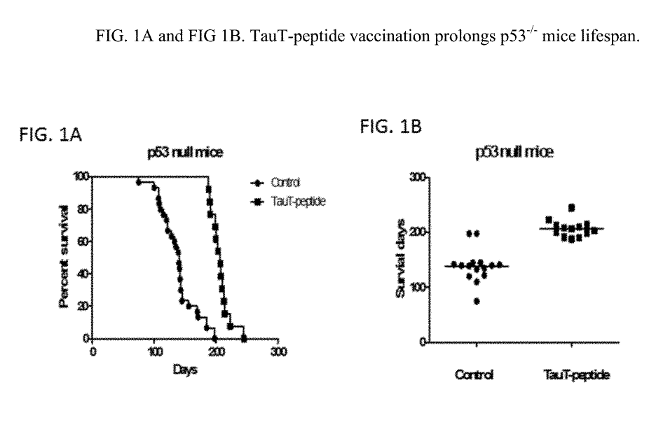

To evaluate the efficacy of TauT-peptide as a cancer vaccine, p53 null (C57BL/6J background) were injected with either TauT-peptide (100 μg/kg) or vehicle only at the age of 4 weeks and boosted 2 weeks later (n=12 for each group). All mice were observed daily for signs of morbidity and the development of tumors. The p53^{-/-} control mice started to develop tumor (mainly lymphoma) and died at the age of 2.5 months. The median lifespan for p53^{-/-} control mice was 4.6 months, which consistent with other studies. The p53^{-/-} mice vaccinated with TauT-peptide vaccine showed 100% response to the vaccine. TauT-peptide vaccination delayed tumor develops in all vaccinated p53^{-/-} mice, which started to develop tumor (mainly lymphoma) and died at the age of 6.3 months. TauT-peptide vaccination significantly increased (1.5 fold) lifespan of the p53^{-/-} mice from 4.6 months (control) to 6.9 months (TauT-peptide vaccinated, FIG. 1). This finding is the first to demonstrate that a peptide vaccine against a specific target of p53 tumor suppressor gene can delay, inhibit and/or treat p53 mutation related spontaneous tumorigenesis.

### Example 2

TauT-Ab Induces Cancer Cell Death In vitro

Human breast cancer cell line MCF-7, human placental carcinoma cell line Jar, human leukemia cell lines, HL-60 and Jurkat, human embryonic kidney (293), pig renal proximal tubular cells (LLC-PK1), and murine fibroblast (10)1 cells were cultured according to AATC (American Association Tissue Culture) guidelines. Briefly, cells were grown as confluent monolayers in 10 cm diameter tissue culture plates in media specific for each cell line with 10% fetal calf serum at 37° C. in the presence of 5% CO₂ in a humidified incubator.

Taurine transport studies were performed on 80% confluent monolayers 2 days after seeding cells. Briefly, cells were washed with Earle's Balanced Salt Solution (EBSS) at 37° C. Uptake was initiated by the addition of uptake buffer (2 mM KCl, mM MgCl₂, 96 mM NaCl, 1.8 mM CaCl₂, 5 mM Hepes, pH 7.6) to which 50 μM unlabeled taurine and 0.5 μCi/ml ¹⁴C-taurine (Perkin Elmer, Boston, Mass.) were added. After incubation for 30 min at room temperature, uptake was terminated by the removal of uptake buffer followed by three rapid washes with cold EBSS. Cells were solubilized in 1% SDS in 0.2 N NaOH and radioactivity was counted in a Packard 2000-CA Liquid Scintillation Analyzer.

TauT-Ab specifically blocks taurine uptake by LLC-PK1 cells. LLC-PK1 cells were treated with TauT-Ab for 30 min and then taurine uptake was measured as described in the Methods. Untreated and pre-immune IgG treated cells were used as controls. In this study, we found that TauT-Ab decreases taurine uptake by LLC-PK1 cells in a dose-dependent manner. In contrast, pre-immune IgG has no effect on taurine uptake by LLC-PK1 cells.

Flow Cytometry-Jurkat and thymocytes were centrifuged at 1000 rpm for 5 min, washed in ice-cold PBS containing 1% bovine serum albumin (BAS, Sigma) in PBS by centrifugation at 1000 rpm, resuspended in 0.5 ml of the same, then fixed with 1 ml of 70% ethanol (−20° C.). The fixed cells were then washed 3 times by centrifuging at 1000 rpm for 10 min and resuspended in 3 ml of the BSA buffer. The washed pellet was then finally resuspended in 1 ml of the BSA buffer to which 100μg/ml of RNAse A was added to remove interfering double stranded RNA, and 5 μg/ml propidium iodide (PI) was added to stain DNA. Cells were incubated at 37° C. for 10-15 min in the dark. Cells were then analyzed using a FACStar Plus flow cytometer (BD Biosciences, MA). Data were analyzed using Cell-Quest software.

Cell death was assayed by TACS Annexin V-FITC kits (R&D system, MN). Cells were collected by centrifugation at 1000 rpm for 5 min at room temperature. Cells were washed by resuspending in PBS and then pellet by centrifugation. Gently resuspend cells in the Annexin V at a concentration of 1×10⁵ cells/100 μl. Incubate in the dark for 15 min at room temperature, and then add 400 μl 1 x binding buffer to each sample. Cells were then analyzed using a FACStar Plus flow cytometer (BD Biosciences, MA). Data were analyzed using Cell-Quest software.

Cell viability was determined by the ratio of life cells out of totally counted cells. Cells cultured in the 96-well plate were trypsinized by using 1x trypsin/EDTA, and then the reaction was stopped by the addition of medium. Equal volume of trypan blue stain (0.4%) was added to the cells. Cells were counted under the microscope using a hemacytometr.

All experiments were performed in triplicate. The data represent the mean±SEM of 3 or 4 experiments. Statistical comparisons were made using one-way ANOVA and Student's t test to determine significant differences in the means.

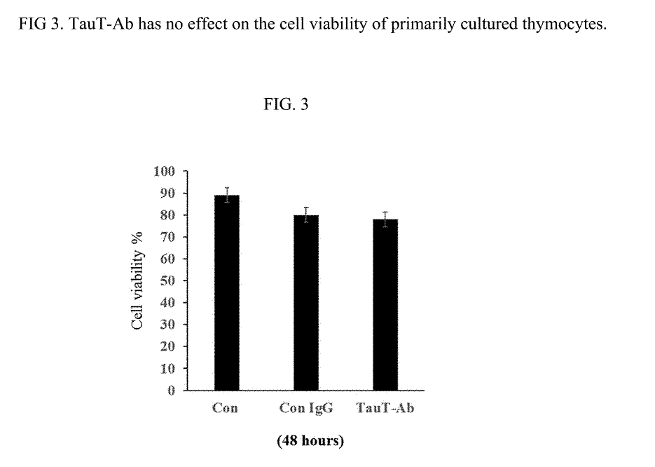

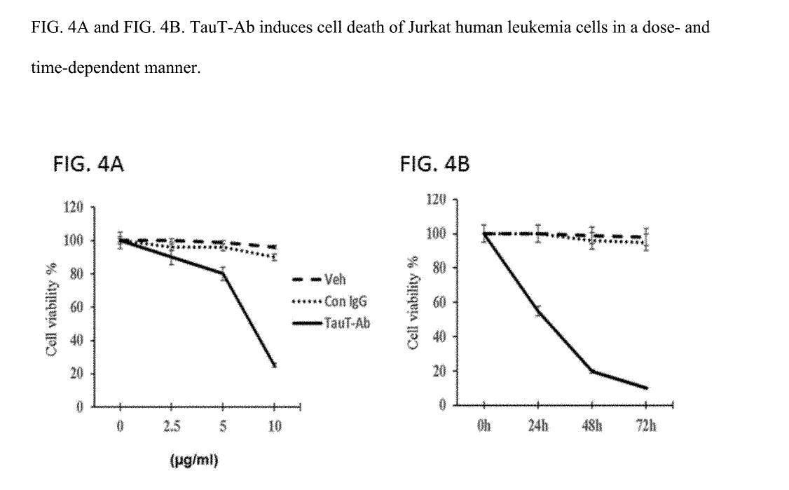

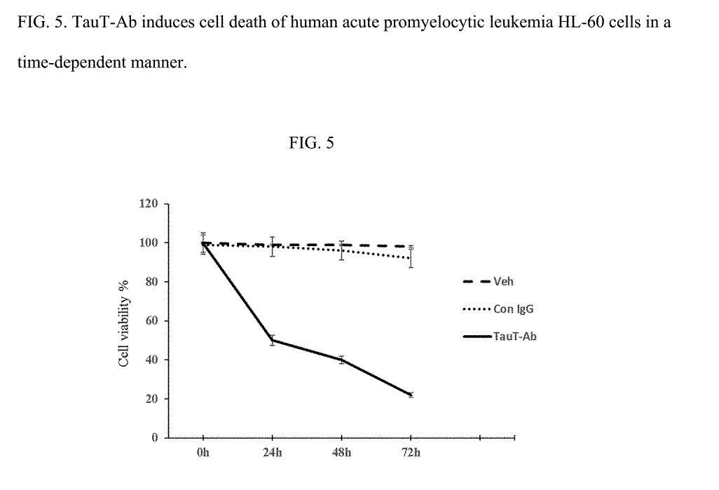

TauT-Ab specifically inhibits the cell growth and induces the death of human leukemia Jurkat and HL-60 cells in a dose-dependent manner (FIGS. 4 and 5). Pre-immune IgG does not induce the death of Jurkat and HL-60 leukemia cells. However, TauT-Ab and pre-immune IgG had no effect on the viability of primarily cultured thymocytes. (FIG. 3). These results suggest that the TauT-Ab targets the leukemia cells by specifically binding to the TauT located on the surface of the leukemia cells. It is important to note that leukemia cells express a very high level of TauT on their membrane surface. In contrast, the primary thymocytes do not express a detectable TauT on the cell surface.

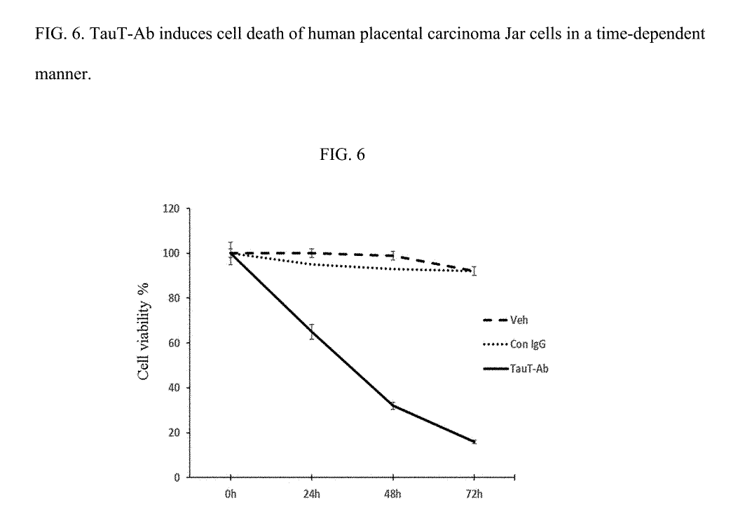

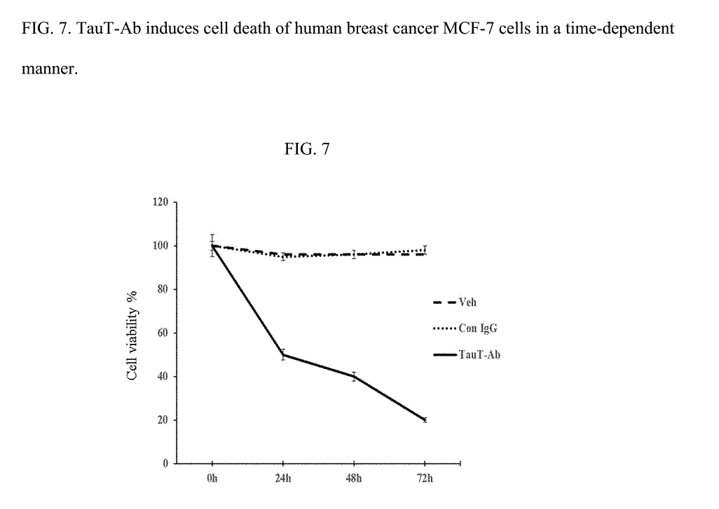

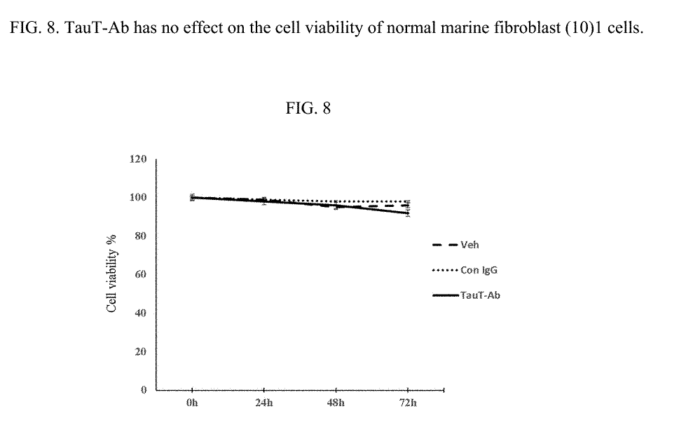

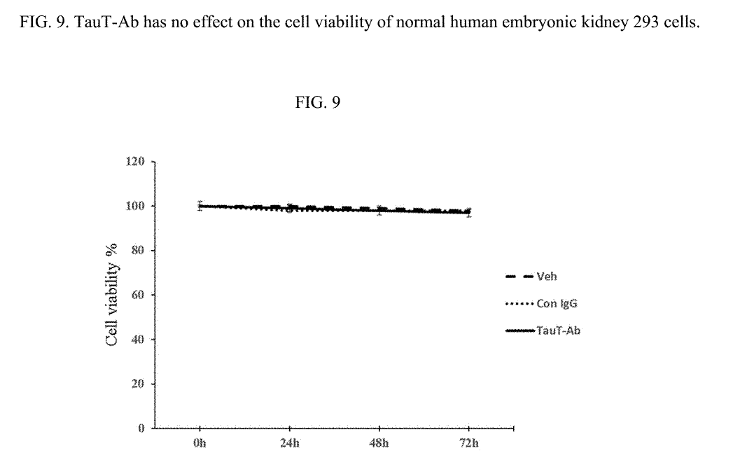

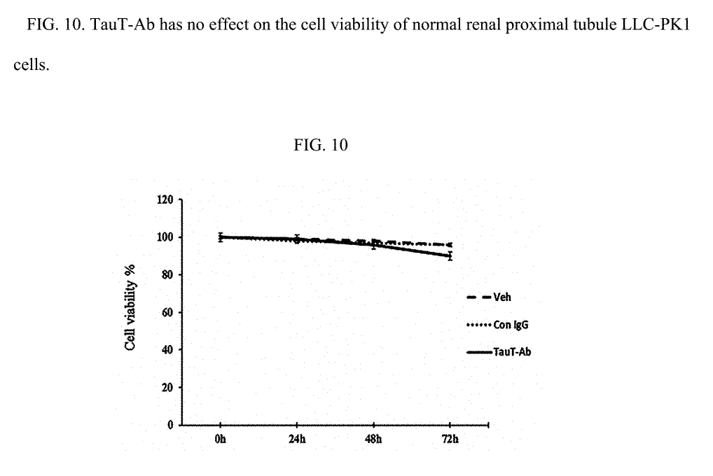

TauT-Ab specifically inhibits cell growth and induces the death of human breast and placental carcinoma cells, since pre-immune IgG did not show any effect on MCF-7 and Jar cells (FIGS. 6 and 7). However, TauT-Ab has little effect on the normal marine fibroblast (10)1 cells, normal human embryonic kidney 293 cells, and normal kidney LLC-PK1 cells (FIG. 8-10), indicating that TauT-Ab can selectively target and kill the tumor cells rather than normal cells.

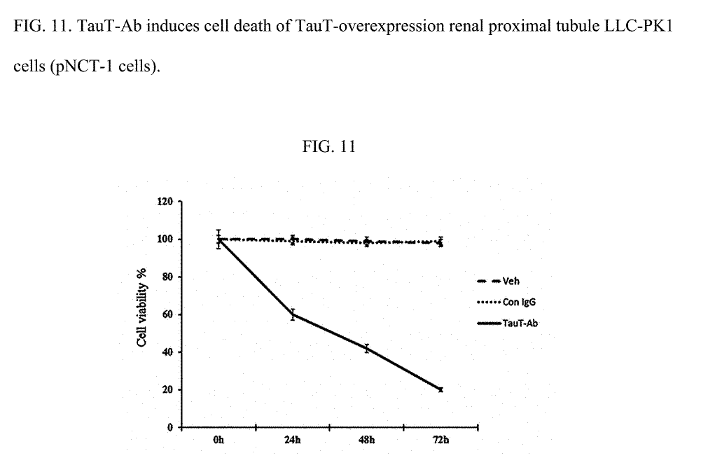

TauT-Ab specifically targets the cells overexpressing TauT on their surface. To determine whether altered expression (overexpression) of TauT on the surface of tumor cells led it to become the target of the TauT-Ab, the TauT gene was stably transfected into LLC-PK1 cells and the cells were treated with TauT-Ab and pre-immune IgG as described above. In this study we found that TauT-Ab inhibits cell growth and induces death of the cells overexpressing TauT, and had little effect on the wild-type LLC-PK1 cells (FIG. 11). This finding suggests that TauT-Ab selectively targets and kills the cells overexpress TauT on their membrane surface.

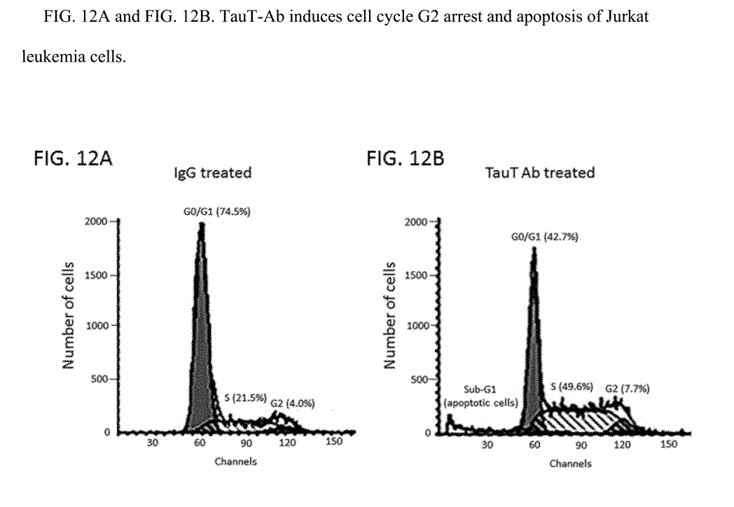

TauT-Ab induces cell cycle arrest of human leukemia Jurkat cells. To study the mechanisms of how TauT-Ab inhibits carcinoma cells proliferation, Jurkat cells were treated with TauT-Ab for 48 hours and cell cycle was analyzed by flow cytometry. Pre-immune IgG was used as negative control. In this study we found that the TauT-Ab induces a G₂M arrest of Jurkat cells, while pre-immune IgG had no effect on cell cycle of Jurkat cells (FIG. 12).

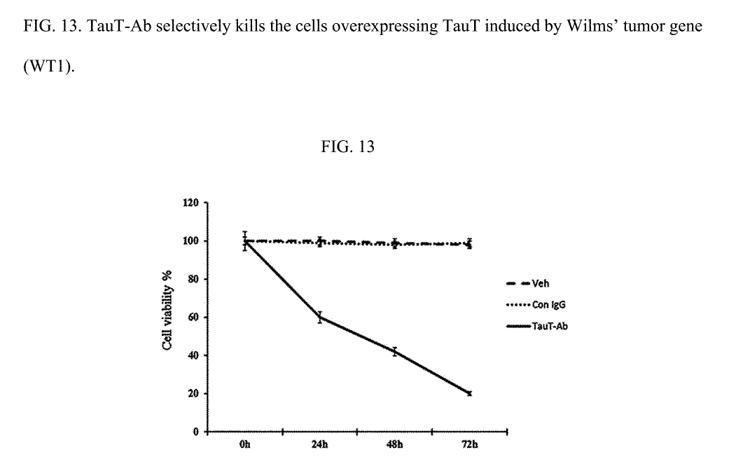

TauT-Ab selectively kills the cells overexpressing TauT induced by Wilms' tumor gene (WT1). In this study WT1 was stably transfected into the LLC-PK1 cells and the cells were named as WT-7. Treatment of WT-7 cells with TauT-Ab inhibits cell growth and induces cells death within 24 hours. In contrast, pre-immune IgG had no effect on the cell viability of WT7 (FIG. 13), indicating that TauT-Ab selectively kills the cells which overexpress a WT1 gene. This finding is very important, since overexpression of WT1 has been found in various types of human leukemia.

### INDUSTRIAL APPLICABILITY

Therapeutic Uses of TauT-peptide and TauT-peptide induced antibody designated as TauT-Ab.

The present invention further relates to TauT-peptide vaccine, -Ab, antibody conjugates, and single-chain immunotoxins reactive with human carcinoma cells, particularly human leukemia cells.

As used in this example, the following words or phrases have the meanings specified.

As used in this example, “joined” means to couple directly or indirectly one molecule with another by whatever means, e.g., by covalent bonding, by non-covalent bonding, by ionic bonding, or by non-ionic bonding. Covalent bonding includes bonding by various linkers such as thioether linkers or thioester linkers. Direct coupling involves one molecule attached to the molecule of interest. Indirect coupling involves one molecule attached to another molecule not of interest which in turn is attached directly or indirectly to the molecule of interest.

As used in this example, “recombinant molecule” means a molecule produced by genetic engineering methods.

As used in this example, “fragment” is defined as at least a portion of the variable region of the immunoglobulin molecule which binds to its target, i.e. the antigen binding region. Some of the constant region of the immunoglobulin may be included.

As used in this example, an “immunoconjugate” means any molecule or ligand such as an antibody or growth factor chemically or biologically linked to a cytotoxin, a radioactive agent, an anti-tumor drug or a therapeutic agent. The antibody or growth factor may be linked to the cytotoxin, radioactive agent, anti-tumor drug or therapeutic agent at any location along the molecule so long as it is able to bind its target. Examples of immuoconjugates include immunotoxins and antibody conjugates.

As used in this example, “selectively killing” means killing those cells to which the antibody binds.

As used in this example, examples of “carcinomas” include bladder, breast, colon, liver, lung, ovarian, placenta, pancreatic carcinomas, and leukemia.

As used in this example, “immunotoxin” means an antibody or growth factor chemically or biologically linked to a cytotoxin or cytotoxic agent.

As used in this example, an “effective amount” is an amount of the antibody, immunoconjugate, recombinant molecule that kills cells or inhibits the proliferation thereof.

As used in this example, “competitively inhibits” means being capable of binding to the same target as another molecule. With regard to an antibody, competitively inhibits mean that the antibody is capable of recognizing and binding the same antigen binding region to which another antibody is directed.

As used in this example, “antigen-binding region” means that part of the antibody, recombinant molecule, the fusion protein, or the immunoconjugate of the invention, which recognizes the target or portions thereof.

As used in this example, “therapeutic agent” means any agent useful for therapy including anti-tumor drugs, cytotoxins, cytotoxic agents, and radioactive agents.

As used in this example, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O, P′-(DDD)), interferons and radioactive agents.

As used in this example, “acytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used in this example, “radioisotope” includes any radioisotope which is effective in destroying a tumor. Examples include, but are not limited to, cobalt-60 and X-rays. Additionally, naturally occurring radioactive elements such as uranium, radium, and thorium which typically -represent mixtures of radioisotopes, are suitable examples of a radioactive agent.

As used in this example, “administering” means oral administration, administration as a suppository, topical contact, intravenous, intraperitoneal, intramuscular or subcutaneous administration, or the implantation of a slow-release device such as a miniosmotic pump, to the subject.

As used in this example, “directly” means the use of antibodies coupled to a label. The specimen is incubated with the labeled antibody, unbound antibody is removed by washing, and the specimen may be examined.

As used in this example, “indirectly” means incubating the specimen with an unconjugated antibody, washing and incubating with a fluorochrome-conjugated antibody. The second or “sandwich” antibody thus reveals the presence of the first.

As used in this example “reacting” means to recognize and bind the target. The binding may be non-specific. Specific binding is preferred.

As used in this example, “curing” means to provide substantially complete tumor regression so that the tumor is not palpable for a period of time, i.e., >/=10 tumor volume doubling delays (TVDD=the time in days that it takes for control tumors to double in size).

As used in this example, “tumor targeted antibody” means any antibody which recognizes the S4 peptide antigen on tumor (i.e., cancer) cells.

As used in this example, “inhibit proliferation” means to interfere with cell growth by whatever means.

As used in this example, “mammalian tumor cells” include cells from animals such as human, ovine, porcine, murine, bovine animals.

As used in this example, “pharmaceutically acceptable carrier” includes any material which when combined with the antibody retains the antibody's immunogenicity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules.

Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

The present invention relates to TauT-Ab that are highly specific for carcinoma cells. More particularly, the antibodies react with a range of carcinomas such as breast, placenta carcinomas, and leukemia while showing none or limited reactivity with normal cells.

The term “TauT-Ab” as used herein includes whole, intact polyclonal and monoclonal antibody materials, and chimeric antibody molecules. The TauT-Ab described above includes any fragments thereof containing the active antigen-binding region of the antibody such as Fab, F(ab′)2 and Fv fragments, using techniques well established in the art (11). The TauT-Ab of the invention also includes fusion proteins.

Also included within the scope of the invention are anti-idiotypic antibodies to the TauT-Ab of the invention. These anti-idiotypic antibodies can be produced using the TauT-Ab and/or the fragments thereof as immunogen and are useful for diagnostic purposes in detecting humoral response to tumors and in therapeutic applications, e.g., in a vaccine, to induce an anti-tumor response in patients (12).

In addition, the present invention encompasses antibodies that are capable of binding to the same antigenic determinant as the TauT-Abs and competing with the antibodies for binding at that site. These include antibodies having the same antigenic specificity as the TauT-Abs but differing in species origin, isotype, binding affinity or biological functions (e.g., cytotoxicity). For example, class, isotype and other variants of the antibodies of the invention having the antigen-binding region of the TauT-Ab can be constructed using recombinant class-switching and fusion techniques (13, 14). Thus, other chimeric antibodies or other recombinant antibodies (e.g., fusion proteins wherein the antibody is combined with a second protein such as a lymphokine or a tumor inhibitory growth factor) having the same binding specificity as the TauT-specific antibodies fall within the scope of this invention.

Genetic engineering techniques known in the art may be used as described herein to prepare recombinant immunotoxins produced by fusing antigen binding regions of TauT-Ab to a therapeutic or cytotoxic agent at the DNA level and producing the cytotoxic molecule as a chimeric protein. Examples of therapeutic agents include, but are not limited to, antimetabolites, alkylating agents, anthracyclines, antibiotics, and anti-mitotic agents. Antimetabolites include methotrexate, 6-mercaptopurine, 6-thioguanine, cytarabine, 5-fluorouracil decarbazine. Alkylating agents include mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin. Anthracyclines include daunorubicin (formerly daunomycin) and doxorubicin (also referred to herein as adriamycin). Additional examples include mitozantrone and bisantrene. Antibiotics include dactinomycin (formerly actinomycin), bleomycin, mithramycin, and anthramycin (AMC). Antimytotic agents include vincristine and vinblastine (which are commonly referred to as vinca alkaloids). Other cytotoxic agents include procarbazine, hydroxyurea, asparaginase, corticosteroids, mytotane (O, P′-(DDD)), interferons. Further examples of cytotoxic agents include, but are not limited to, ricin, doxorubicin, taxol, cytochalasin B, gramicidin D, ethidium bromide, etoposide, tenoposide, colchicin, dihydroxy anthracin dione, 1-dehydrotestosterone, and glucocorticoid.

Clearly analogs and homologs of such therapeutic and cytotoxic agents are encompassed by the present invention. For example, the chemotherapuetic agent aminopterin has a correlative improved analog namely methotrexate. Further, the improved analog of doxorubicin is a Fe-chelate. Also, the improved analog for 1-methylnitrosourea is lomustine. Further, the improved analog of vinblastine is vincristine. Also, the improved analog of mechlorethamine is cyclophosphamide.

Recombinant immunotoxins, particularly single-chain immunotoxins, have an advantage over drug/antibody conjugates in that they are more readily produced than these conjugates, and generate a population of homogenous molecules, i.e. single peptides composed of the same amino acid residues. The techniques for cloning and expressing DNA sequences encoding the amino acid sequences corresponding to TauT single-chain immunotoxins, e.g synthesis of oligonucleotides, PCR, transforming cells, constructing vectors, expression systems, and the like are well-established in the art, and most practitioners are familiar with the standard resource materials for specific conditions and procedures.

The following include preferred embodiments of the immunoconjugates of the invention. Other embodiments which are known in the art are encompassed by the invention. The invention is not limited to these specific immunoconjugates, but also includes other immunoconjugates incorporating antibodies and/or antibody fragments according to the present invention.

The conjugates comprise at least one drug molecule connected by a linker of the invention to a targeting ligand molecule that is reactive with the desired target cell population. The ligand molecule can be an immunoreactive protein such as an antibody, or fragment thereof, a non-immunoreactive protein or peptide ligand such as bombesin or, a binding ligand recognizing a cell associated receptor such as a lectin or steroid molecule.

Further, because the conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin, a protein such as tumor necrosis factor, alpha-interferon, beta-interferon, nerve growth factor, platelet derived growth factor, tissue plasminogen activator, or, biological response modifiers such as, for example, lymphokines, interleukin-I (“IL1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

The preferred drugs for use in the present invention are cytotoxic drugs, particularly those that are used for cancer therapy. Such drugs include, in general, alkylating agents, anti-proliferative agents, tubulin binding agents and the like. Preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. As noted previously, one skilled in the art may make chemical modifications to the desired compound in order to make reactions of that compound more convenient for purposes of preparing conjugates of the invention.

As noted, one skilled in the art will appreciate that the invention also encompasses the use of antigen recognizing immunoglobulin fragments. Such immunoglobulin fragments may include, for example, the Fab′, F(ab′)2, F[v ] or Fab fragments, or other antigen recognizing immunoglobulin fragments. Such immunoglobulin fragments can be prepared, for example, by proteolytic enzyme digestion, for example, by pepsin or papain digestion, reductive alkylation, or recombinant techniques. The materials and methods for preparing such immunoglobulin fragments are well-known to those skilled in the art (15).

The immunoglobulin can be a “chimeric antibody” as that term is recognized in the art. Also, the immunoglobulin may be a “bifunctional” or “hybrid” antibody, that is, an antibody which may have one arm having a specificity for one antigenic site, such as a tumor associated antigen while the other arm recognizes a different target, for example, a hapten which is, or to which is bound, an agent lethal to the antigen-bearing tumor cell. Alternatively, the bifunctional antibody may be one in which each arm has specificity for a different epitope of a tumor associated antigen of the cell to be therapeutically or biologically modified. In any case, the hybrid antibodies have a dual specificity, preferably with one or more binding sites specific for the hapten of choice or one or more binding sites specific for a target antigen, for example, an antigen associated with a tumor, an infectious organism, or other disease state.

Biological bifunctional antibodies are described previous (16). Such hybrid or bifunctional antibodies may be derived, as noted, either biologically, by cell fusion techniques, or chemically, especially with cross-linking agents or disulfide bridge-forming reagents, and may be comprised of whose antibodies and/or fragments thereof. Particularly preferred bifunctional antibodies are those biologically prepared from a “polydome” or “quadroma” or which are synthetically prepared with cross-linking agents such as bis-(maleimideo)-methyl ether (“BMME”).

In addition the immunoglobulin may be a single chain antibody (“SCA”). These may consist of single chain Fv fragments (“scFv”) in which the variable light (“V[L]”) and variable heavy (“V[H]”) domains are linked by a peptide bridge or by disulfide bonds. Also, the immunoglobulin may consist of single V[H ]domains (dAbs) which possess antigen-binding activity (17).

Especially preferred for use in the present invention is chimeric monoclonal antibodies, preferably those chimeric antibodies having specificity toward a tumour-associated antigen. As used in this example, the term “chimeric antibody” refers to a monoclonal antibody comprising a variable region, i.e. binding region, from one source or species and at least a portion of a constant region derived from a different source or species, usually prepared by recombinant DNA techniques. Chimeric antibodies comprising a murine variable region and a human constant region are preferred in certain applications of the invention, particularly human therapy, because such antibodies are readily prepared and may be less immunogenic than purely murine monoclonal antibodies. Such murine/human chimeric antibodies are the product of expressed immunoglobulin genes comprising DNA segments encoding murine immunoglobulin variable regions and DNA segments encoding human immunoglobulin constant regions. Other forms of chimeric antibodies encompassed by the invention are those in which the class or subclass has been modified or changed from that of the original antibody. Such “chimeric” antibodies are also referred to as “class-switched antibodies”. Methods for producing chimeric antibodies involve conventional recombinant DNA and gene transfection techniques now well known in the art (18).

Encompassed by the term “chimeric antibody” is the concept of “humanized antibody”, that is those antibodies in which the framework or “complementarity” determining regions (“CDR”) have been modified to comprise the CDR of an immunoglobulin of different specificity as compared to that of the parent immunoglobulin. In a preferred embodiment, a murine CDR is grafted into the framework region of a human antibody to prepare the “humanized antibody” (19). Particularly preferred CDR′S correspond to those representing sequences recognizing the antigens noted above for the chimeric and bifunctional antibodies.

One skilled in the art will recognize that a bifunctional-chimeric antibody can be prepared which would have the benefits of lower immunogenicity of the chimeric or humanized antibody, as well as the flexibility, especially for therapeutic treatment, of the bifunctional antibodies described above. Such bifunctional-chimeric antibodies can be synthesized, for instance, by chemical synthesis using cross-linking agents and/or recombinant methods of the type described above. In any event, the present invention should not be construed as limited in scope by any particular method of production of an antibody whether bifunctional, chimeric, bifunctional-chimeric, humanized, or an antigen-recognizing fragment or derivative thereof.

In addition, the invention encompasses within its scope immunoglobulins (as defined above) or immunoglobulin fragments to which are fused active proteins, for example, an enzyme of the type previously disclosed.

As noted, “bifunctional”, “fused”, “chimeric” (including humanized), and “bifunctional-chimeric” (including humanized) antibody constructions also include, within their individual contexts constructions comprising antigen-recognizing fragments. As one skilled in the art will recognize, such fragments could be prepared by traditional enzymatic cleavage of intact bifunctional, chimeric, humanized, or chimeric-bifunctional antibodies. If, however, intact antibodies are not susceptible to such cleavage, because of the nature of the construction involved, the noted constructions can be prepared with immunoglobulin fragments used as the starting materials; or, if recombinant techniques are used, the DNA sequences, themselves, can be tailored to encode the desired “fragment” which, when expressed, can be combined in vivo or in vitro, by chemical or biological means, to prepare the final desired intact inumunoglobulin “fragment”. It is in this context, therefore, that the term “fragment” is used.

Furthermore, as noted above, the immunoglobulin (antibody), or fragment thereof, used in the present invention may be polyclonal or monoclonal in nature. Monoclonal antibodies are the preferred immunoglobulins, however. The preparation of such polyclonal or monoclonal antibodies now is well known to those skilled in the art who, of course, are fully capable of producing useful immunoglobulins which can be used in the invention (20). In addition, hybridomes and/or monoclonal antibodies which are produced by such hybridomas and which are useful in the practice of the present invention are publicly available from sources such as the American Type Culture Collection (“ATCC”).

Particularly preferred antibodies for use in the present invention are those which recognize tumor associated antigens (SEQ ID NO 3).

Therapeutic Applications of TauT-Abs

The properties of the TauT-Ab suggest a number of in vivo therapeutic applications.

First, the TauT-Ab can be used alone to target and kill tumor cells in vivo. The antibody can also be used in conjunction with an appropriate therapeutic agent to treat human carcinoma. For example, the antibody can be used in combination with standard or conventional treatment methods such as chemotherapy, radiation therapy or can be conjugated or linked to a therapeutic drug, or toxin, as well as to a lymphokine or a tumour-inhibitory growth factor, for delivery of the therapeutic agent to the site of the carcinoma.

Techniques for conjugating such therapeutic agents to antibodies are well known (21).

Alternatively, the TauT-peptide vaccine or TauT-Ab can be coupled to high-energy radiation, e.g., a radioisotope such as ¹³¹I, when localized at the tumor site, results in a killing of several cell diameters. According to yet another embodiment, the TauT-Ab can be conjugated to a second antibody to form an antibody heteroconjugate for the treatment of tumor cells as described previously (22).

Still other therapeutic applications for the TauT-Ab of the invention include conjugation or linkage, e.g., by recombinant DNA techniques, to an enzyme capable of converting a prodrug into a cytotoxic drug and the use of that antibody-enzyme conjugate in combination with the prodrug to convert the prodrug to a cytotoxic agent at the tumor site (23).

Still another therapeutic use for the TauT-Ab involves use, either in the presence of complement or as part of an antibody-drug or antibody-toxin conjugate, to remove tumor cells from the bone marrow of cancer patients. According to this approach, autologous bone marrow may be purged ex vivo by treatment with the antibody and the marrow infused back into the patient (24).

Furthermore, chimeric TauT, recombinant immunotoxins and other recombinant constructs of the invention containing the specificity of the antigen-binding region of the C35 monoclonal antibody, as described earlier, may be used therapeutically. For example, the single-chain immunotoxins of the invention, may be used to treat human carcinoma in vivo.

Similarly, a fusion protein comprising at least the antigen-binding region of the TauT-Ab joined to at least a functionally active portion of a second protein having anti-tumor activity, e.g., a lymphokine or oncostatin can be used to treat human carcinoma in vivo. Furthermore, recombinant techniques known in the art can be used to construct bispecific antibodies wherein one of the binding specificities of the antibody is that of TauT, while the other binding specificty of the antibody is that of a molecule other than TauT.

Finally, anti-idiotypic antibodies of the TauT-Ab may be used therapeutically in active tumor immunization and tumor therapy (25).

The present invention provides a method for selectively killing tumor cells expressing the antigen that specifically binds to the TauT-Ab or functional equivalent. This method comprises reacting the immunoconjugate (e.g. the immunotoxin) of the invention with said tumor cells. These tumor cells may be from a human carcinoma.

In accordance with the practice of this invention, the subject may be a human, equine, porcine, bovine, murine, canine, feline, and avian subjects. Other warm blooded animals are also included in this invention.

The present invention also provides a method for curing a subject suffering from a cancer. The subject may be a human, dog, cat, mouse, rat, rabbit, horse, goat, sheep, cow, chicken. The cancer may be identified as a breast, bladder, retinoblastoma, papillary cystadenocarcinoma of the ovary, Wilm's tumor, or small cell lung carcinoma and is generally characterized as a group of cells having tumor associated antigens on the cell surface. This method comprises administering to the subject a cancer killing amount of a tumor targeted antibody joined to a cytotoxic agent. Generally, the joining of the tumor targeted antibody with the cytotoxic agent is made under conditions which permit the antibody so joined to bind its target on the cell surface. By binding its target, the tumor targeted antibody acts directly or indirectly to cause or contribute to the killing of the cells so bound thereby curing the subject.

Also provided is a method of inhibiting the proliferation of mammalian tumor cells which comprises contacting the mammalian tumor cells with a sufficient concentration of the immunoconjugate of the invention so as to inhibit proliferation of the mammalian tumor cells.

The subject invention further provides methods for inhibiting the growth of human tumor cells, treating a tumor in a subject, and treating a proliferative type disease in a subject. These methods comprise administering to the subject an effective amount of the composition of the invention.

It is apparent therefore that the present invention encompasses pharmaceutical compositions, combinations and methods for treating human carcinomas. For example, the invention includes pharmaceutical compositions for use in the treatment of human carcinomas comprising a pharmaceutically effective amount of a TauT-Ab and a pharmaceutically acceptable carrier.

The compositions may contain the TauT-peptide, TauT-Ab or antibody fragments, either unmodified, conjugated to a therapeutic agent (e.g., drug, toxin, enzyme or second antibody) or in a recombinant form (e.g., chimeric TauT, fragments of chimeric TauT, bispecific TauT or single-chain immunotoxin TauT). The compositions may additionally include other antibodies or conjugates for treating carcinomas (e.g., an antibody cocktail).

The TauT-peptide vaccine, TauT-Ab, antibody conjugate and immunotoxin compositions of the invention can be administered using conventional modes of administration including, but not limited to, intravenous, intraperitoneal, oral, intralymphatic or administration directly into the tumor. Intravenous administration is preferred.

The compositions of the invention may be in a variety of dosage forms which include, but are not limited to, liquid solutions or suspension, tablets, pills, powders, suppositories, polymeric microcapsules or microvesicles, liposomes, and injectable or infusible solutions. The preferred form depends upon the mode of administration and the therapeutic application.

The compositions of the invention also preferably include conventional pharmaceutically acceptable carriers and adjuvants known in the art such as human serum albumin, ion exchangers, alumina, lecithin, buffer substances such as phosphates, glycine, sorbic acid, potassium sorbate, and salts or electrolytes such as protamine sulfate.

The most effective mode of administration and dosage regimen for the compositions of this invention depends upon the severity and course of the disease, the patient's health and response to treatment and the judgment of the treating physician. Accordingly, the dosages of the compositions should be titrated to the individual patient.

The molecules described herein may be in a variety of dosage forms which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The most effective mode of administration and dosage regimen for the molecules of the present invention depends upon the location of the tumor being treated, the severity and course of the cancer, the subject's health and response to treatment and the judgment of the treating physician. Accordingly, the dosages of the molecules should be titrated to the individual subject.

The interrelationship of dosages for animals of various sizes and species and humans based on mg/kg of surface area is described previously (26).

### CITATION LIST

### Patent Document

Patent Document 1: US2016009799 (A1)

### Non-patent Document

1. Han X, Chesney R W. Knockdown of TauT expression impairs human embryonic kidney 293 cell development. Advances in experimental medicine and biology. 2013;776:307-20. doi: 10.1007/978-1-4614-6093-0_28. PubMed PMID: 23392892.

2. Han X, Patters A B, Chesney R W. Transcriptional repression of taurine transporter gene (TauT) by p53 in renal cells. The Journal of biological chemistry. 2002;277(42):39266-73. doi: 10.1074/jbc.M205939200. PubMed PMID: 12163498.

3. Hollstein M, Sidransky D, Vogelstein B, Harris C C. p53 mutations in human cancers. Science. 1991;253(5015):49-53. PubMed PMID: 1905840.

4. Han X, Budreau A M, Chesney R W. Role of conserved peptide in taurine transporter inactivation modulated by protein kinase C. Journal of the American Society of Nephrology:JASN. 1996;7(10):2088-96. PubMed PMID: 8915968.

5. Houghten R A. General method for the rapid solid-phase synthesis of large numbers of peptides: specificity of antigen-antibody interaction at the level of individual amino acids. Proceedings of the National Academy of Sciences of the United States of America. 1985;82(15):5131-5. PubMed PMID: 2410914; PMCID: PMC390513.

6. Hoffman S L, Berzofsky J A, Isenbarger D, Zeltser E, Majarian W R, Gross M, Ballou W R. Immune response gene regulation of immunity to Plasmodium berghei sporozoites and circumsporozoite protein vaccines. Overcoming genetic restriction with whole organism and subunit vaccines. Journal of immunology. 1989;142(10):3581-4. PubMed PMID: 2497175.

7. Tainer J A, Getzoff E D, Alexander H, Houghten R A, Olson A J, Lerner R A, Hendrickson W A. The reactivity of anti-peptide antibodies is a function of the atomic mobility of sites in a protein. Nature. 1984;312(5990):127-34. PubMed PMID: 6209578.

8. Clarke B E, Newton S E, Carroll A R, Francis M J, Appleyard G, Syred A D, Highfield P E, Rowlands D J, Brown F. Improved immunogenicity of a peptide epitope after fusion to hepatitis B core protein. Nature. 1987;330(6146):381-4. doi: 10.1038/330381a0. PubMed PMID: 2446137.

9. Wahl R L, Parker C W, Philpott G W. Improved radioimaging and tumor localization with monoclonal F(ab′)2. J Nucl Med. 1983;24(4):316-25. PubMed PMID: 6339689.

10. Han X, Budreau A M, Chesney R W. Ser-322 is a critical site for PKC regulation of the MDCK cell taurine transporter (pNCT). Journal of the American Society of Nephrology:JASN. 1999;10(9):1874-9. PubMed PMID: 10477138.

11. Rousseaux J, Rousseaux-Prevost R, Bazin H. Optimal conditions for the preparation of proteolytic fragments from monoclonal IgG of different rat IgG subclasses. Methods Enzymol. 1986;121:663-9. PubMed PMID: 3088386.

12. Nepom G T, Nelson K A, Holbeck S L, Hellstrom I, Hellstrom K E. Induction of immunity to a human tumor marker by in vivo administration of anti-idiotypic antibodies in mice. Proceedings of the National Academy of Sciences of the United States of America. 1984;81(9):2864-7. PubMed PMID: 6609369; PMCID: PMC345172.

13. Thammana P, Scharff M D. Immunoglobulin heavy chain class switch from IgM to IgG in a hybridoma. European journal of immunology. 1983;13(8):614-9. doi: 10.1002/eji.1830130803. PubMed PMID: 6884421.

14. Neuberger M S, Williams G T, Fox R O. Recombinant antibodies possessing novel effector functions. Nature. 1984;312(5995):604-8. PubMed PMID: 6095112.

15. Lamoyi E, Nisonoff A. Preparation of F(ab′)2 fragments from mouse IgG of various subclasses. J Immunol Methods. 1983;56(2):235-43. PubMed PMID: 6402547.

16. Menard S, Canevari S, Colnaghi M I. Hybrid antibodies in cancer diagnosis and therapy. Int J Biol Markers. 1989;4(3):131-4. PubMed PMID: 2693537.

17. Winter G, Milstein C. Man-made antibodies. Nature. 1991;349(6307):293-9. doi: 10.1038/349293a0. PubMed PMID: 1987490.

18. Morrison S L, Johnson M J, Herzenberg L A, Oi V T. Chimeric human antibody molecules: mouse antigen-binding domains with human constant region domains. Proceedings of the National Academy of Sciences of the United States of America. 1984;81(21):6851-5. PubMed PMID: 6436822; PMCID: PMC392030.

19. Mage R G. Designing antibodies for human therapies. Nature. 1988;333(6176):807-8. doi: 10.1038/333807c0. PubMed PMID: 3386732.

20. Kohler G, Milstein C. Continuous cultures of fused cells secreting antibody of predefined specificity. Nature. 1975;256(5517):495-7. PubMed PMID: 1172191.

21. Thorpe P E, Ross W C. The preparation and cytotoxic properties of antibody-toxin conjugates. Immunol Rev. 1982;62:119-58. PubMed PMID: 7042536.

22. Paya C V, McKean D J, Segal D M, Schoon R A, Showalter S D, Leibson P J. Heteroconjugate antibodies enhance cell-mediated anti-herpes simplex virus immunity. Journal of immunology. 1989;142(2):666-71. PubMed PMID: 2536060.

23. Senter P D. Activation of prodrugs by antibody-enzyme conjugates: a new approach to cancer therapy. FASEB journal:official publication of the Federation of American Societies for Experimental Biology. 1990;4(2):188-93. PubMed PMID: 2404820.

24. Majdic O, Sugita K, Touw I, Koller U, Stockinger H, Delwel R, Lowenberg B, Knapp W. Bone-marrow purging with monoclonal antibodies and human complement in ALL and AML. Haematol Blood Transfus. 1987;31:86-8. PubMed PMID: 3327791.

25. Pan Y, Yuhasz S C, Amzel L M. Anti-idiotypic antibodies: biological function and structural studies. FASEB journal:official publication of the Federation of American Societies for Experimental Biology. 1995;9(1):43-9. PubMed PMID: 7821758.

26. Freireich E J, Gehan E A, Rall D P, Schmidt L H, Skipper H E. Quantitative comparison of toxicity of anticancer agents in mouse, rat, hamster, dog, monkey, and man. Cancer Chemother Rep. 1966;50(4):219-44. PubMed PMID: 4957125.

What is claimed is:

1. An polypeptide, which consist of at most 14 contiguous amino acid residues of a mammalian (including human) TauT protein.

2. The polypeptide of claim 1, which consists of SEQ ID NO 3.

3. The polypeptide of claims 1 and 2, which is designated as TauT-peptide and is used to prepare a TauT-peptide cancer vaccine.

4. A composition suitable for pharmaceutical use, comprising the polypeptide of claims 1 to 3 and a pharmaceutically acceptable carrier.

5. The composition of claim 4, wherein the pharmaceutically acceptable carrier is an adjuvant.

6. A method of prevent and treatment of cancers with TauT-peptide cancer vaccine by inducing the antibody against TauT (designated as TauT-Ab), comprising administering an effective amount of the TauT-peptide vaccine of claim 1-5 to cancer patients.

7. The antibody of claim 6, which binds to TauT that is characterized by the following functions: Inhibits the growth of tumor cells, and induces apoptosis of tumor cells.


SEQUENCE LISTING (3 sequences)

SEQ ID NO: 1 - DNA, 2864 bp, Homo sapiens
  ref <301>: AUTHORS: Ramamoorthy,S., Leibach,F.H., Mahesh,V.B., Han,H., Yang-Feng,T., Blakely,R.D. and Ganapathy,V.
  ref <302>: TITLE: Functional characterization and chromosomal localization of a cloned taurine transporter from human placenta
  ref <303>: JOURNAL: Biochem. J.
  ref <304>: VOLUME: 300
  ref <305>: ISSUE: 3
  ref <306>: PAGES: 893-900
  ref <307>: DATE: 1994-10-18
  ref <308>: DATABASE ACCESSION NUMBER: U16120
  ref <309>: DATABASE ENTRY DATE: 1994-10-25
  ref <313>: RELEVANT RESIDUES IN SEQ ID NO: (1)..(2864)
gcccaaagaactcaaacaaatttacaagaaacaaacaaccccatcaacaagtggccgaagatatgaacagacacgtctcagaagaaggcatttatggagacaacagacacgcgaggtcaggaagaagccgcttataaattaccgcttccttcgcgccgccgccaacgccgagccccgaggaccgcaagcccagaggacaagctgcgccaagagggagtgcggacgttcacccagcgggtcagagagcgagcgggcaggcagcccccggccggcggaacccggcacagccgagcagagcgcgggcggcgccgcagccaccccagatccagaaccagaaccacagcccttctgaggagctcccaaacaaagcaaggagatggccaccaaggagaagctgcagtgtctgaaagatttccacaaggacatcctgaagccctcaccagggaagagcccaggcaagcggcctgaggacgaggctgagggaaaacctccgcagagggagaagtggtctagcaagatcgactttgtgctctctgtggctggcggcttcgtgggcttgggcaacgtctggcgcttcccgtacctctgctacaagaatggtggaggtgcgtttctcataccgtattttattttcctgtttgggagcggcctgcctgtgtttttcttggagatcatcataggccagtacacctctgaagggggcatcacctgctgggaaaagatctgccccttgttctctggtatcggctatgcctccgttgtaattgtgtccctcctgaatgtctactacatcgtcatcctggcctgggccacatactacctgttccagtccttccagaaggagctgccctgggcacactgcaaccacagctggaacacacctcactgcatggaggacaccatgcgcaagaacaagagtgtctggatcaccatcagctccaccaacttcacctcccctgtcatcgagttctgggagcgcaacgtgctgagcttgtcccctggaatcgaccacccaggctctctgaaatgggacctcgctctctgccttcttttagtctggctagtgtgtttcttctgcatctggaagggcgtcaggtccactgggaaggtcgtctacttcacagccacttttccattcgccatgctcctggtgctgctggtccgagggctgacgctgccgggcgcgggcgcaggcatcaagttctatctgtatcctgacatcacccgccttgaggacccacaggtgtggattgacgctgggactcagatattcttctcttatgccatctgcctgggggctatgacctcgctggggagctacaacaagtacaagtataactcgtacagggactgtatgctgctgggatgcctgaacagtggtaccagttttgtgtctggcttcgcaattttttccatcctgggcttcatggcacaagagcaaggggtggacattgctgatgtggctgagtcaggtcctggcctggccttcattgcctacccaaaagctgtgacaatgatgccgctgcccacattttggtccattcttttttttattatgcttctcttgcttggactggatagccagtttgttgaagttgaaggacagatcacatccttggttgatctttacccatccttcctaaggaagggttatcgtcgggaaatcttcatcgccttcgtgtgtagcatcagctacctgctggggctgacgatggtgacggagggtggcatgtatgtgtttcagctctttgactactatgcagctagcggtgtatgccttttgtgggttgcattctttgaatgttttgttattgcctggatatatggaggtgataacctttatgatggtattgaggacatgattggctatcggcccgggccctggatgaagtacagctgggctgtgatcactccagttctctgtgttggatgtttcatcttctcgctcgtcaagtacgtacccctgacctacaacaaaacatacgtgtaccccaactgggccattgggctgggctggagcctggccctttcctccatgctctgcgttcccttggtcatcgtcatccgcctctgccagactgaggggccgttccttgtgagagtcaagtacctgctgaccccaagggaacccaaccgctgggctgtggagcgcgagggagccacaccttacaactctcgcaccgtcatgaacggcgctctcgtgaaaccgacccacatcattgtggagaccatgatgtgagctctctcgggtcgacggggccaacggtttcctgctgtttactaacattagattctcataggaccaggtttacagagctttatatttgcactaggattttttttttttgtaattgtcacagaaaatgtaattgtgggtgtgtgtgcgtgcgtgtgtgtgtgtgtgtgtatcgtgtgtgtgtgttttgttttgatttgggggatattttgtacaaaaagaaaacccacgggaagatgtccgtggagaggcagagctttcatactgaattagatgtattttatgggaatttggtaaatttttctttgtattttttttttttacatataagtatatatacacttagagattgtcatatacttttaccacttgaattgatcttcttgccagcaatagatctcattttcaaaagcaattcttcggtctgtgtagctggcagaaagttctgtccagtaaacgcaggatggaattttcctgggactctacacccatcttaaggtggtataccttccaaatcctggttcagatggaagaaatagcaggagagaggacccattagctggcagacccagggggaagaaaggagggctgtgaggagatacctcattaaacttggcttagtgaagag

SEQ ID NO: 2 - protein, 620 aa, Home sapiens [sequence omitted]

SEQ ID NO: 3 - protein, 14 aa, Home sapiens
  TauT PEPTIDE (1)..(14)
GSYNKYKYNSYRDC